(19) United States
(12) Patent Application Publication
(10) Pub. No.: US 2004/0037429 A1
(43) Pub. Date: Feb. 26, 2004

(54) Stethoscope

(76) Inventor: Victor A. Candioty, Oak Park, CA (US)

Correspondence Address: MATTHEW A. NEWBOLES, STETINA BRUNDA GARRED & BRUCKER, SUITE 250, 75 ENTERPRISE, ALISO VIEJO, CA, 92656, US

(21) Appl. No.: 10/226,875

(22) Filed: Aug. 23, 2002

(51) Int. Cl. A61B 7/04; A61F 11/06; G10K 11/16; H03B 29/00

(52) U.S. Cl. 381/67; 381/71.6

(57) ABSTRACT

An improved stethoscope for use in environments with high ambient noise levels. The stethoscope incorporates active noise cancellation via destructive interference to attenuate the unwanted ambient noise. Once the active noise cancellation wave is generated to cancel the unwanted ambient noise, the stethoscope is operative, per conventional stethoscopes, to isolate the desired sounds sought to be heard emanating from the body.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention relates generally to an improved stethoscope, and more particularly, an electronic stethoscope utilizing active noise cancellation technology in order to isolate sounds generated from within a patient's body from those of the surrounding environment. In this regard, the stethoscope of the present invention is particularly useful in environments having high ambient noise levels, as is frequently encountered on rescue helicopters, light aircraft, ground-based ambulances, and combat situations.

The construction and usage of stethoscopes are well-known. Essentially, the conventional stethoscope consists of a two-sided headpiece with a diaphragm and bell on opposing surfaces, tubing, and ear pieces. The diaphragm comprises a thin plastic covering utilized to listen to high pitched sounds. Typically, on the opposed side of the diaphragm, is a bell utilized to listen to low pitched sounds. Extending from the diaphragm (and bell) is flexible plastic tubing, which may take the form of either a single tube or double tubes, that branch out for positioning upon opposed sides of the wearer's head. Formed on the proximal-most ends of such tubing are ear pieces designed to fit into the wearer's ears.

In use, stethoscopes facilitate the ability to hear sounds within the body emanating from the heart, lungs and intestinal tract, and are further utilized for measuring blood pressure. Generally, the sounds said to be heard via the stethoscope can range from about 20 to 1,000 Hz for heart sounds and about 70 to 2,000 Hz for breath sounds. Through the use of well-known diagnostic techniques, such sounds are carefully evaluated to confirm whether the same are indicative of a particular condition.

Despite its usefulness as a non-invasive diagnostic tool to help localize a variety of conditions, conventional stethoscopes are poorly suited for use in areas having high ambient noise levels. In this respect, it is virtually impossible to utilize a conventional stethoscope on rescue helicopters, ground-based ambulances and the like where external noise substantially interferes with the ability to hear sound emanating from within the body. While electronic stethoscopes have been developed to enable health care workers to hear heart and lung sounds clearly in high noise environments, typically via the use of direct amplification combined with either fixed and/or adjustable filters, the same are incapable of fluidly countering fluctuations in ambient noise, such as through sudden changes in frequency and/or volume, to thus enable internal body sounds to be isolated in a manner capable of being optimally perceived. As a consequence, the condition of a particular patient may be improperly assessed or otherwise delayed, which can, and frequently does, cause the patient's condition to worsen.

Accordingly, there is a substantial need in the art for an improved stethoscope that is capable of effectively identifying sounds emanating from within a patient's body in an environment having a high ambient noise level in a manner superior to prior art stethoscopes. There is additionally a substantial need in the art for such a stethoscope that can be utilized in a high ambient noise environment that is capable of identifying and isolating those sounds emanating from within the body while concurrently suppressing or substantially eliminating competing sound, and in particular sound having an overlapping frequency range as those sounds sought to be heard. There is likewise a need in the art for such a stethoscope that is capable of fluidly countering ambient noise levels that continuously change in terms of frequency and volume. There is yet further a need for such a stethoscope that is capable of being constructed utilizing conventional technology, can be constructed at relatively low cost, is exceptionally more effective and efficient than prior art stethoscopes, particularly with respect to usage in high ambient noise environments, can be configured to be operated easily and quickly, and can in all other respects be utilized as per conventional stethoscopes.

### BRIEF SUMMARY OF THE INVENTION

The present invention specifically addresses and alleviates the above-identified deficiencies in the art. In this regard, the present invention is directed to an electronic stethoscope which incorporates active noise cancellation to thus enable the stethoscope to isolate sound emanating within the body to thus enable the same to be heard in an environment having high to extremely high ambient noise levels.

According to a preferred embodiment, the stethoscope utilizes a first headpiece microphone operative to be positioned against a patient's body to hear sounds emanating therefrom, as per conventional stethoscopes, but also will be operative to pick up unwanted ambient noise of the surrounding environment. As a consequence a signal is produced that is indicative of both the sounds generating within the patient's body and the surrounding environment, which may be amplified and filtered to produce a resultant signal.

At least one second microphone is provided that is positioned in relatively close proximity to the diaphragm microphone to record the ambient noise in the surrounding environment, preferably to the same intensity as that recorded by the diaphragm microphone.

The output of the first and the second microphone are concurrently fed to a microprocessor integrated within the ambient noise-cancellation device that in turn executes a spectral subtraction algorithm that is operative to produce a cancelling anti-noise signal based on the output of the second microphone that is equal to, but 180° out of phase, with the ambient noise component received from the diaphragm microphone. Such anti-noise signal is amplified and transmitted to the headset of the stethoscope of the present invention, which in essence is introduced to the stethoscope wearer such that it matches the ambient noise of the surrounding environment. In use, the ambient noise of the environment and the anti-noise signal played to the headset of the stethoscope, cancel each other out, effectively removing the substantial portion of the noise energy from the environment. As a consequence, unwanted noise is suppressed and the desired sound, whether it be a heart sound, lung sound, etc., is ultimately transmitted to the stethoscope wearer's ears. To better achieve that end, it is contemplated that the resultant signal produced from the stethoscope which is ultimately transmitted in audible form to the stethoscope wearer may also be processed further to enhance the ability thereof to be heard. In this respect, it is contemplated that such signal may undergo further processing, such as filtering, amplification, and the like to thus enable the body sounds to be heard in as clear and accurate form as possible to allow for optimal assessment of the patient.

The stethoscope of the present invention will further preferably include either a battery power source or operate via a conventional alternating current. It is further contemplated that the stethoscope, and in particular the electronic components thereof, will be durable and encased in waterproof, chemically impervious materials insofar as the same are intended to be deployed in rugged terrain, such as combat conditions and the like, and must be capable of functioning in extreme environmental conditions, such as extreme heat and cold.

### BRIEF DESCRIPTION OF THE DRAWINGS

These as well as other features of the present invention will become more apparent upon reference to the drawings wherein:

FIG. 1 is a partial perspective view of a health care provider utilizing a stethoscope constructed in accordance with a preferred embodiment of the present invention to assess the condition of a patient.

FIG. 2 is a schematic diagram of an ambient noise cancellation device integrated within the stethoscope of the present invention for suppressing ambient noise in the surrounding environment within which the patient is assessed.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

The present invention is directed to an improved electronic stethoscope capable of being utilized in areas having high to extremely high ambient noise levels while still effectively isolating and conveying clear and accurate sounds emanating from within the body of a patient. In this regard, the stethoscope of the present invention is particularly well suited for use in the field, particularly with respect to emergency medicine, where a patient's condition must be assessed and evaluated amidst considerable ambient noise. Exemplary of such conditions include accidents, natural disasters, and combat/terrorist situations where care must be administered amidst extreme conditions, such as patient transport aboard ambulances and aircraft, and particularly helicopters, which generate substantial operating noise and can typically involve the extensive use of sirens.

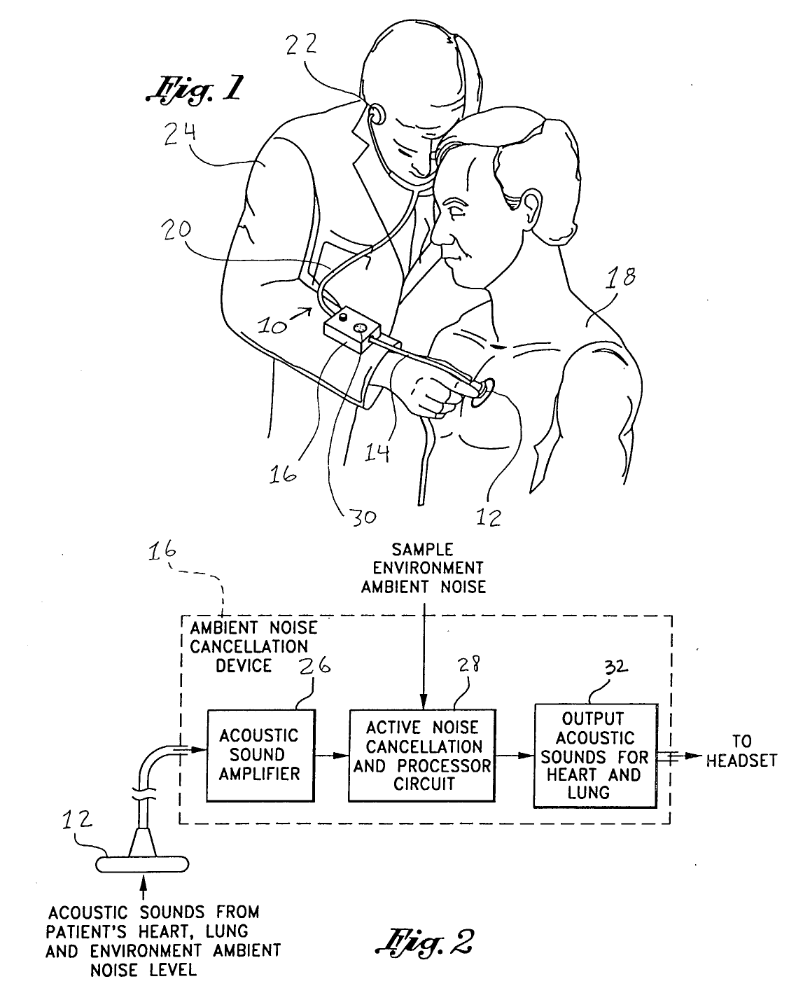

Referring now to FIG. 1, there is perspectively illustrated the stethoscope 10 of the present invention as constructed according to a preferred embodiment. As shown, the stethoscope 10 included a first headpiece microphone 12, which is preferably configured as per conventional headpiece elements utilized in conventional stethoscopes, that is coupled to tubing 14, the latter for relaying sounds emanating from within the patient. The tubing 14 is coupled to an ambient noise cancellation device 16, discussed more fully below, which is operative to facilitate the removal of unwanted ambient noise emanating from the external environment about the patient 18. A signal is relayed through further tubing 20 and ultimately to earphones or headset 22 worn by the health care provider 24. In this respect, it should be recognized at the outset that the stethoscope of the present invention is intended to function in all respects like a conventional stethoscope and relay all sounds emanating within the body as may be perceived through the use of conventional stethoscopes to thus make conventional diagnoses.

In order to provide the ability of the stethoscope to be utilized in those areas including high ambient noise, however, the stethoscope 10 of the present invention incorporates electronics operative to provide active noise cancellation to cancel unwanted ambient noise. To achieve that end, the first microphone 12 is operative to transmit a signal indicative of a particular sound emanating from within the body, which will encompass all sounds within at least 20-2,000 MHz, As illustrated in FIG. 2. Such microphone 12, however, will necessarily also record sounds from the ambient environment, which may have a considerably higher volume and include sound having a frequency overlapping with that sought to be heard emanating from the patient. Such signal may be processed further, and specifically may be sent through a high-gain amplifier 26, as further shown in FIG. 2, as well as a low-pass filter (not shown) which are preferably included as part of the ambient noise cancellation device 16.

To eradicate the unwanted sound emanating from the environment, the stethoscope 10 of the present invention employs active noise cancellation 28 within the ambient noise cancellation device 16 that is operative to duplicate the unwanted sounds of the ambient environment such that the same are emitted out of phase with the unwanted ambient sound to thus produce a destructive acoustic interference. In this regard, it is contemplated that existing active noise cancellation technology known in the art can be readily deployed in the practice of the present invention.

In the embodiment shown in FIGS. 1-2, the ambient noise cancellation component of the stethoscope of the present invention will preferably employ a second microphone 30 working in tandem with the first microphone 12 to sample the ambient noise from the environment. Such second microphone 30 is operative to transmit a signal indicative of the ambient noise, which is preferably at the same intensity level as that received from the first microphone 12, but will omit any sort of sound component indicative of those sounds emanating from a patient's body sought to be isolated. In this regard, and as will be appreciated by those skilled in the art, to the extent that the patient's heart and lung sounds were to be concurrently transmitted to both microphones, the active noise cancellation system component of the stethoscope of the present invention would be operative to cancel the same.

By means of active noise cancellation 28, implemented via a processor circuit, noise is thus reduced by generation of a cancelling anti-noise signal which is equal to, but 180° out of phase with the ambient noise component present in the signal produced from the first microphone 12. As discussed above, such active noise cancellation is accomplished through conventional noise cancellation technology known to those skilled in the art.

The output signal 32 will thus possess a component indicative of the anti-noise that is introduced to the stethoscope 10 via the headset worn by the wearer of the stethoscope. In this respect, such anti-noise will be delivered at a decibel level matching that of the ambient noise level in the surrounding environment. The two respective sound sources, namely the noise of the ambient environment and the anti-noise generated through the headphones 22 of the stethoscope 10 of the present invention, effectively cancel each other out, removing a significant portion of the noise energy from the environment.

As a result, a final audible component is generated via output 32 indicative of the sound emanating from the patient that is then ultimately fed to the headphone set formed as part of the stethoscope that will be placed upon the wearer's head. To the extent desired, such output signal 32 may be processed further, either by filtering, amplification, and the like, such that the same may be more clearly heard by the stethoscope wearer. In this regard, it is contemplated that the headphone set will completely encircle the ears to thus insulate the same as much as possible. As is well-known, fully covering the ear has the advantage of not only blocking out the extreme ambient noise produced from the outside environment (passive noise reduction), but will also enable the active noise cancellation mechanisms of the present invention to better deliver the anti-noise wave through the speakers disposed in the headset 22 of the stethoscope to thus cancel out ambient noise leaving the remaining signal indicative of the sounds emanating from the patients's body to be heard. Such audible signal may be assessed as per conventional sounds emanating from the body to thus enable the health care provider to make an informed diagnosis.

In further refinements of the present invention, it is contemplated that the stethoscope 10 may include either an internal battery source, as will be necessary to operate the processor unit and other electronic components utilized in the functioning of the stethoscope, or may also be adapted to run on an alternating current. It is additionally contemplated that the stethoscope 10 will be formed of durable materials and capable of operating in extreme environments, particularly with respect to temperature extremes in both hot and cold, dusty and windy conditions, and conditions involving corrosive and toxic chemicals. It is further contemplated that such stethoscope 10 may be fabricated to be waterproof in nature to thus enable the same to be utilized in boat rescue operations or other situations requiring immediate medical attention to be provided near a body of water, whether it be an ocean, lake, river or the like. Still further, it is contemplated that the stethoscope 10 of the present invention should be able to withstand extreme differences in pressure, and will preferably be constructed for use in high altitude applications.

Advantageously, by virtue of the fact that the stethoscope of the present invention effectively cancels out ambient noise, the stethoscope 10 of the present invention allows the sought-after sounds emanating from the body to be heard and evaluated in a conventional manner. In this regard, it is contemplated that the stethoscope 10 of the present invention will be capable of providing information consistent with conventional stethoscopes and thus will not require special medical evaluation or interpretation of sound heard through such stethoscope 10. Accordingly, conventional diagnoses may be made based upon those sounds observed through the stethoscope 10 of the present invention without the need for any further technical consideration.

Of further advantage, the active noise cancellation system 28 as incorporated within the stethoscope 10 of the present invention is operative to cancel noise that may fluctuate in terms of volume and frequency. Other electronic stethoscopes, in contrast, merely utilize filters and the like that, although effective in blocking sounds having a particular frequency, do not provide a consistent noise cancellation in environments where noise can fluctuate substantially in terms of both volume and frequency, frequently encountered, particularly with respect to emergency rescues in combat situations.

As will be appreciated, additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. A stethoscope for use in isolating and accurately conveying sounds emanating from within a person located in an environment having a high ambient noise level comprising:
a) a first microphone operative to be positioned against the person for generating a signal indicative at least one sound emanating from within the person and the ambient noise of the environment;
b) at least one second microphone operative to produce a signal indicative of the ambient noise of the environment; and
c) an ambient noise cancellation device operatively coupled to said at least one second microphone and said headphones, said active noise cancellation system being operative to generate a signal substantially out of phase with a portion of said signal generated by said diaphragm microphone corresponding to said ambient noise of the environment; and
d) a headphone set operative to receive and audibly transmit said signals received from said diaphragm microphone and said ambient noise cancellation device.

2. The stethoscope of claim 1 wherein said ambient noise cancellation device generates a signal that is approximately 180° out of phase with said portion of said signal generated from said first microphone corresponding to the ambient noise of the environment.

3. The stethoscope of claim 1 wherein said ambient noise cancellation device comprises a processor unit operative to execute a spectral subtraction algorithm, said algorithm being operative to generate said signal being substantially out of phase with said portion of said signal generated from said first microphone corresponding to of said ambient noise of said environment.

4. The stethoscope of claim 1 further comprising:
a) a battery power source operatively coupled to said first microphone, said at least one second microphone, said ambient noise cancellation device, and said headset for providing power respectively thereto.

5. The stethoscope of claim 1 wherein said stethoscope further comprises an apparatus for coupling to a power source.

6. The stethoscope of claim 1 wherein said stethoscope is formed from a water impervious material.

7. The stethoscope of claim 1 wherein such stethoscope is capable of functioning at 32° F. or less.

8. The stethoscope of claim 1 wherein such stethoscope is capable of functioning at 100° F. or higher.

9. Wherein said diaphragm microphone is operative to detect body sounds ranging from about 20 to 2000 Hz.
